(19) United States
(12) Patent Application Publication
(10) Pub. No.: US 2003/0094503 A1
(43) Pub. Date: May 22, 2003

(54) Dual reservoir dispenser for an air freshener or insecticide

(76) Inventors: Shaun Patrick Rymer, Beverley (GB); Clare Louise Robinson, Hull (GB)

Correspondence Address: FISH & RICHARDSON P.C., 45 ROCKEFELLER PLAZA, SUITE 2800, NEW YORK, NY, 10111, US

(21) Appl. No.: 09/989,552

(22) Filed: Nov. 20, 2001

(51) Int. Cl. A61L 9/04; A01G 27/00

(52) U.S. Cl. 239/34; 239/145; 239/6

(57) ABSTRACT

In an apparatus for dispensing a volatile substance from a main reservoir, a second reservoir is provided which contains an additional quantity of the volatile substance. The substance is dispensed from the second reservoir during a delay period in which it cannot be dispensed from the main reservoir.

The present invention relates to an apparatus for dispensing a volatile composition in the vapour phase. In particular, the present invention relates to an apparatus for dispensing an air freshener or insecticide in the vapour phase.

Various devices for dispensing volatile compositions such as air fresheners and insecticides are known. For example, the volatile composition may be impregnated onto a solid carrier, from which it gradually evaporates and enters the atmosphere. Alternatively, the composition may be provided in the form of a liquid, for example, as a thickened liquid or gel. Such liquids are generally contained in a reservoir, formed, at least in part, of a material that is permeable to the vapours of the composition. As the liquid evaporates, the vapours of the composition can permeate through the material and enter the surrounding atmosphere.

A gas may also be used to aid the dispersion of volatile compositions into the atmosphere. For example, the dispensing device may include an electrochemical cell for producing a gas at a controlled rate. As more gas is produced, the gas pressure in the device increases. This build-up of pressure is used to transport the composition into the atmosphere. The rate at which the gas is generated can be used to control the rate at which the composition is released into the atmosphere. WO 96/41159 describes a cell that is suitable for producing a gas for dispensing fluid media. In use, the cell produces a surge of gas, which moves a piston to push the fluid out of the dispenser.

It has now been found that there can be an initial delay before the gas pressure generated by the electrochemical cell is sufficiently large to transport the volatile composition into the atmosphere. We have found that the problem is only significant in applications where the rate of gas production is necessarily low. This is the case for air freshener and insecticide applications, where the composition has to be released gradually over a prolonged period of time. A low rate of gas production is required to ensure that the product is released at the desired rate. At the same time, however, this low rate results in a delay before any product is dispensed.

According to the present invention, there is provided an apparatus for dispensing a volatile composition into the atmosphere, the apparatus comprising:

means for generating a gas;

a first reservoir containing a first volatile composition;

a second reservoir containing a second volatile composition; and

means for actuating the second reservoir to dispense the volatile composition contained in the second reservoir from the apparatus;

the apparatus being such that in use, gas generated by the means for generating a gas acts on the first volatile composition to dispense the volatile composition from the first reservoir.

Preferably, the means for generating a gas is an electrochemical cell.

In use, the electrochemical cell is actuated to generate a gas, which accumulates in the first reservoir. As a result of this accumulation, the gas pressure in the first reservoir increases, pushing the first volatile composition out of the apparatus, for example, onto an emanator, from which it evaporates and into the atmosphere. In general, there is an initial delay of, for example, 12 or more hours before the gas pressure in the apparatus is sufficient to push the composition out of the apparatus. Thus, to ensure that the composition can be dispensed from the apparatus during this initial delay period, the second reservoir is actuated to release its contents into the atmosphere. In a preferred embodiment, the second reservoir is provided with a seal that may be ruptured or removed to allow the composition to escape, for example, by evaporation.

The first reservoir may take the form of, for example, a tubular housing. This first reservoir may be provided with at least one opening, from which the first composition can escape. Preferably, a plurality of openings are provided. The total cross-sectional area of the opening(s) may be 0.01 to 1.2 mm², preferably, 0.1 to 0.5 mm², for example, 0.1 to 0.3 mm². Each opening may be defined by a micro-channel, which may or may not be substantially circular in cross section. The cross-sectional area of each micro-channel may be 0.01 to 0.4 mm², preferably, 0.05 to 0.1 mm², more preferably, 0.07 to 0.1 mm². In a preferred embodiment, 1 to 7, preferably, 2 to 5, for example, 3 openings are provided. In order to prevent the first reservoir from releasing the air freshener or insecticidal composition prematurely, the opening or openings of the reservoir may be provided with a seal or cover, which may be ruptured or removed when the apparatus is ready for use. For example, the seal may be in the form of a piece of metal foil or plastics material, which may be peeled off or ruptured in a convenient manner, for example, by applying finger pressure or by using a pin or the like. In a preferred embodiment, the seal is formed from a sheet of polyethylene laminated onto a metal such as aluminium.

In one embodiment, the opening or openings of the first reservoir are located at one end of the reservoir. Thus, the electrochemical cell may be positioned at or adjacent the opposite end of the reservoir, such that in use, the gas generated by the cell can act on the volatile composition and push it towards the other end of the reservoir and out of the opening(s). Preferably, the gas acts directly on the volatile composition. Thus, in this preferred embodiment, intervening members such as pistons or diaphragms are not required to push the volatile composition out of the apparatus. However, intervening members may be used if desired.

The second reservoir may also take the form of, for example, a tubular housing. The second reservoir may be in fluid communication with the first reservoir. In such an embodiment, it may only be necessary to preload one of the reservoirs with the volatile composition prior to use. For example, it may be possible to preload the first reservoir with the volatile composition and allow some of the volatile composition to flow from the first reservoir into the second, for example, under gravity. In an alternative embodiment, the first and second reservoirs are not in fluid communication with one another. It may therefore be necessary to pre-load both reservoirs with the volatile composition prior to use. In general, the same volatile composition is contained in both reservoirs. However, it is possible to use the same volatile composition in different physical forms. For example, the volatile composition in the first reservoir may be in liquid form, whilst the volatile composition in the second reservoir may be in solid form, or in the form of a concentrated vapour.

The second reservoir may be provided with at least one opening, from which the volatile composition is allowed to escape into the atmosphere. This opening is preferably provided with a seal, which can be released to dispense volatile composition into the atmosphere. For example, the seal may be in the form of a piece of metal foil or plastics material, which may be peeled off or ruptured in a convenient manner, for example, by applying finger pressure or by using a pin or the like. In a preferred embodiment, the seal is formed from a sheet of a polymer, such as polyethylene, laminated onto a metal such as aluminium. The same seal may be employed to seal the opening(s) of both the first and second reservoirs. Thus, by removing the seal on the second reservoir, the compositions from both reservoirs can be released.

Preferably, the volume of the second reservoir is substantially less than that of the first reservoir. For example, the volume of the second reservoir may be 0.1 to 10%, preferably, 0.5 to 2% of the volume of the first reservoir. In one embodiment, the second reservoir contains sufficient composition to ensure that the composition can be dispensed from the apparatus during the initial delay period. In a preferred embodiment, the second reservoir becomes substantially depleted once the gas pressure in the apparatus is sufficient to transport the volatile substance from the first reservoir.

Any suitable electrochemical cell for generating a gas may be employed in the apparatus of the present invention. Examples of suitable cells are described in WO 96/41159. In a preferred embodiment, the electrochemical cell takes the form of a disc, which is positioned adjacent or in the first reservoir.

Any suitable gas can be generated to transport the volatile composition from the apparatus. Examples include carbon dioxide, oxygen and, preferably, hydrogen.

Preferably, the electrochemical cell is capable of generating gas at a rate of 0.001 to 0.5 ml per 24 hour period, preferably, 0.1 to 0.3 ml per 24 hour period. The current generated by the cell may range from 0.08 and 0.8 mA, preferably, 0.14 and 0.4 mA. Preferably, when the apparatus is in operation, the current generated by the cell is carefully controlled to produce the gas at an appropriate rate, to ensure that the volatile composition is dispensed from the apparatus steadily over time.

The apparatus may further comprise a protective casing to protect at least one of the components of the apparatus from damage. In one embodiment, the protective casing surrounds the first and/or the second reservoir. Preferably, the protective casing may also surround the electrochemical cell. The protective casing is generally provided with one or more apertures (eg vents) through which the vapours of the volatile composition may flow into the atmosphere.

In a preferred embodiment, the apparatus is also provided with one or more emanators for aiding the dispersion of the volatile composition into the atmosphere. In use, the volatile composition, for example, from the first reservoir is allowed to drip onto the emanator, from which it is evaporated into the atmosphere. The emanator may take the form of a membrane that is permeable to the volatile composition, allowing the composition to diffuse into the atmosphere. Such membranes may be formed of polyethylene or paper, and are generally thin, so as not to impeded the flow of vapour into the atmosphere. For example, the membrane may have a thickness of 20 to 100 microns, preferably, 30 to 70 microns, and especially 40 to 60 microns. Suitable membranes are described in U.S. Pat. No. 4,634,614 and WO 98/23304.

In order to prevent the apparatus from releasing the volatile composition before it is used, the membrane may be covered with an impermeable sheet that can be removed when the apparatus is ready for use. The sheet may be made of a laminate, such as polyethylene laminated with a metal such as aluminium.

The apparatus of the present invention is particularly suitable for dispensing an air freshener or insecticidal composition. Such compositions may be in the form of a non-thickened or thickened liquid, or gel. Suitable compositions are described in WO 01/00304.

Preferably, the liquid has a relative density of 0.5 to 1.2, preferably, 0.7 to 1.0, more preferably, 0.89 to 0.98. Preferably, also the air freshener or insecticidal composition has a flash point of 50 to 110 deg C., preferably, 60 to 95 deg C., more preferably, 65 to 88 deg C.

The first reservoir preferably has a capacity of 5 to 20 ml, preferably, 10 to 15 ml, more preferably, 12 to 14 ml, for example, 14.5 ml. The reservoir may be filled with 3 to 18 ml, preferably, 10 to 12 ml of the first volatile composition prior to use.

The second reservoir preferably contains 0.05 to 2 ml, preferably, 0.1 to 1 ml, more preferably, 0.4 to 0.6 ml of the composition, before use.

According to a further aspect of the present invention, there is provided a method for dispensing a volatile composition into the atmosphere, the method comprising:

providing an apparatus as herein described;

actuating the second reservoir to release the volatile composition contained in the second reservoir into the atmosphere;

connecting the anode and the cathode of the electrochemical cell to a closed external circuit to generate the gas, if necessary by support of a source of direct current; and

allowing the gas to act on the volatile composition in the first reservoir to dispense the volatile composition into the atmosphere.

In yet another aspect, the present invention provides the use of a second reservoir containing a volatile composition, in an apparatus for dispensing a volatile composition from a main reservoir, said use being for dispensing volatile composition into the atmosphere during a delay period in which volatile composition contained in the main reservoir cannot be dispensed.

These and other aspects of the present invention will now be described by way of example with reference to the drawings in which:

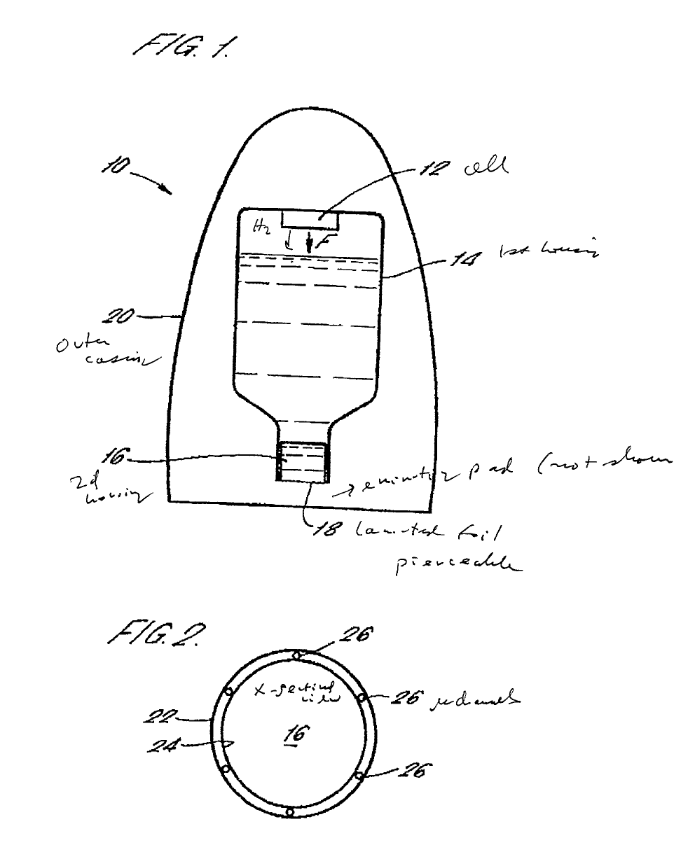

FIG. 1 is a schematic diagram of a preferred embodiment of the invention,

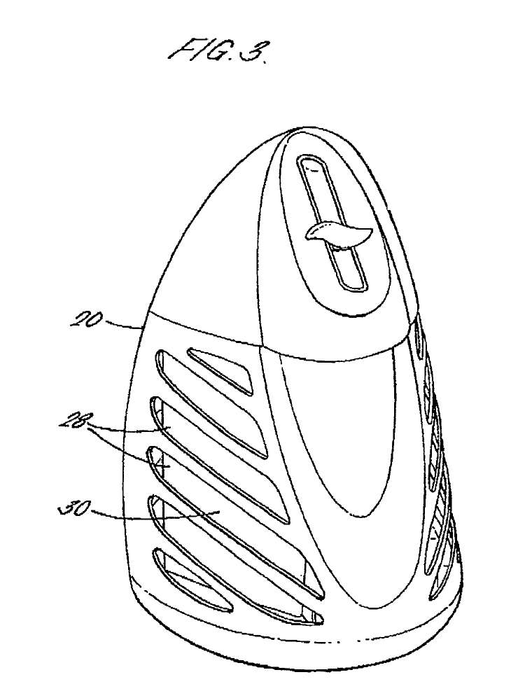

FIG. 2 is a schematic, cross sectional view of the second housing of FIG. 1, and

FIG. 3 is a schematic view of the outer casing of FIG. 1.

Referring first to FIG. 1, the diagram depicts an apparatus 10 for dispensing a liquid air freshener into the atmosphere. The apparatus 10 comprises an electrochemical cell 12, a first housing 14 and a second housing 16. Both housings 14, 16 contain the air freshener to be dispensed. The second housing 16 is sealed with a sheet 18 of laminated foil, which can be pierced to release its contents into the atmosphere. The apparatus further comprises an outer casing 20.

In use, the electrochemical cell 12 is actuated to generate hydrogen, which accumulates in the first housing 14. As the gas pressure in the first housing 14 increases, a downward force is exerted on the air freshener, as indicated by the arrow F. This downward force pushes the air freshener out of the first housing 14 and onto an emanating pad (not shown), from which it evaporates into the surrounding atmosphere.

When the electrochemical cell 12 is first actuated, however, there is an initial delay before the gas pressure is sufficient to force the air freshener out of the first housing 14. To ensure that air freshener can be dispensed from the apparatus 10, therefore, the sheet 18 of the laminated foil is pierced, for example, with a pin to release the contents of the second housing 16. The air freshener contained in the second housing 16 drips onto the emanating pad (not shown) under gravity, and evaporates into the atmosphere.

Reference is now made to FIG. 2, which depicts a cross-sectional view of the second housing 16. The second housing is defined by an outer wall 22 and an inner wall 24. Between the outer and inner walls 22, 24, are located a plurality of micro-channels 26, each having a cross-sectional area of 0.099 mm² The micro-channels serve as openings to connect the interior of the first housing 14 to the outside. When the apparatus 10 is in use, the downward force F causes the air freshener to flow from the interior of the first housing 14 out through the micro-channels 26.

Reference is now made to FIG. 3, which depicts the outer casing 20 of FIG. 1 in greater detail. The outer casing 20 is provided with a series of vents 28, which allow the vapours of the air freshener to flow from the interior of the apparatus 10 to the outside. The interior of the outer casing 20 is lined with a membrane 30 formed of porous paper. The membrane is permeable to the vapours of the air freshener, and aid in the dispersion of these vapours into the atmosphere.

We claim:

1. An apparatus for dispensing a volatile composition into the atmosphere, the apparatus comprising:
means for generating a gas;
a first reservoir containing a first volatile composition;
a second reservoir containing a second volatile composition; and
means for actuating the second reservoir to dispense the volatile composition contained in the second reservoir from the apparatus;
the apparatus being such that, in use, gas generated by the means for generating a gas acts on the first volatile composition to dispense the volatile composition from the first reservoir.

2. An apparatus as claimed in claim 1, wherein the means for generating a gas is an electrochemical cell.

3. An apparatus as claimed in claim 2, wherein the first reservoir is provided with a plurality of openings for releasing the volatile composition contained therein into the atmosphere.

4. An apparatus as claimed in claims 2 or 3, wherein the means for actuating the second reservoir comprises a seal.

5. An apparatus as claimed in claim 4, wherein the seal is in the form of a sheet of metal and/or plastics material, which can be removed or ruptured to release the volatile composition from the apparatus.

6. An apparatus as claimed in claim 5, wherein the seal is also used to seal at least one of the openings of the first reservoir.

7. An apparatus as claimed in claim 2, wherein the volume of the second reservoir is 0.1 to 10% of the volume of the first reservoir.

8. An apparatus as claimed in claim 2 wherein the electrochemical cell is capable of generating gas at a rate of 0.001 to 0.5 ml per 24 hour period.

9. An apparatus as claimed in claim 8, wherein the current generated by the electrochemical cell is 0.08 to 0.8 mA.

10. An apparatus as claimed in claim 2, wherein each of the first and second volatile compositions is an air freshener composition.

11. An apparatus as claimed in claim 2, wherein each of the first and second volatile compositions is an insecticidal composition.

12. An apparatus as claimed in claims 10 or 11, wherein the first volatile composition has a relative density of 0.5 to 1.2 and a flash point of 50 to 110 deg C.

13. An apparatus as claimed in claims 10 or 11, wherein the second volatile composition has a relative density of 0.5 to 1.2 and a flash point of 50 to 110 deg C.

14. A method for dispensing a volatile composition into the atmosphere, the method comprising:
providing an apparatus as claimed in claim 2;
actuating the second reservoir to release the volatile composition contained in said second reservoir into the atmosphere;
connecting the anode and the cathode of the electrochemical cell to a closed external circuit to generate the gas, and
allowing the gas to act on the volatile composition in the first reservoir to dispense said volatile composition into the atmosphere.

15. A method as claimed in claim 14, wherein the second volatile composition is released before the first volatile composition is dispensed.

16. A method as claimed in claims 14 or 15 where in the electrochemical cell is supported by a source of direct current.
